(19) United States
(12) Patent Application Publication
(10) Pub. No.: US 2014/0209119 A1
(43) Pub. Date: Jul. 31, 2014

(54) SURGICAL TOOL CLEANER AND BONE STORAGE DEVICE

(71) Applicant: John F. Fleming, Delray Beach, FL (US)

(72) Inventor: John F. Fleming, Delray Beach, FL (US)

(21) Appl. No.: 14/167,670

(22) Filed: Jan. 29, 2014

Related U.S. Application Data
(60) Provisional application No. 61/758,515, filed on Jan. 30, 2013.

(51) Int. Cl. B08B 1/00

(52) U.S. Cl. CPC B08B 1/002
USPC 134/6; 15/21.1

(57) ABSTRACT

A method and system for cleaning a surgical tool and storing bone and tissue. The system may include a housing including one or more entry openings and one or more brush assembly chambers, each brush assembly chamber in communication with an entry opening, a brush assembly disposed within each brush assembly chamber, a storage container in communication with each brush assembly chamber, and a ramp face that defines the one or more entry opening. The method may include inserting a distal end of a surgical device with biological material into an entry opening of a collection device, rotating a brush assembly within a brush assembly chamber, the brush assembly and brush assembly chamber being disposed within the collection device, the brush assembly chamber being in communication with the entry opening, and removing biological material from the distal end of the surgical device with the rotating brush assembly.

### CROSS-REFERENCE TO RELATED APPLICATION

This application is related to and claims priority from U.S. Provisional Application Ser. No. 61/758515, filed Jan. 30, 2013, entitled KERRISON RONGEUR SURGICAL TOOL CLEANER AND BONE STORAGE DEVICE, the entirety of which is incorporated herein by reference.

### STATEMENT REGARDING FEDERALLY SPONSORED RESEARCH OR DEVELOPMENT

n/a

### FIELD OF THE INVENTION

The present invention relates to a method and system for cleaning a surgical tool and storing bone and soft tissue.

### BACKGROUND OF THE INVENTION

Many medical procedures require the removal of bone and tissue from the patient using a surgical tool. For example, spinal procedures such as spinal fusion, revision procedures, foraminectomy, and laminectomy may involve the removal of bone and/or tissue from the patient. Although any of a variety of specialized tools may be used, bone collection is often performed using a rongeur (also referred to as a Kerrison Rongeur), that generally includes a sharp, scoop-shaped tip adapted to gauge out bone fragments. A rongeur may also be used for other types of surgeries, including maxillofacial and hand surgery.

During currently known procedures involving a rongeur, the surgeon will collect or harvest fragments of bone and tissue from the patient using the distal end, or “collection end,” of the rongeur. The end of the tool is then held away from the patient and rubbed with, for example, one or more pieces of laparotomy gauze (often referred to as a “lap sponge”) or cheesecloth-like gauze by either the surgeon or the surgical assistant. Thus, the bone and tissue fragments are removed from the rongeur and temporarily held as a wad within the gauze. As the procedure continues, more bone and tissue is added to the wad within the gauze. By the end of the procedure, the collected material is mashed together in heap, which material has been exposed to air and contaminants for the duration of the procedure. Additionally, some of the collected bone and tissue is lost, as it often falls out of the gauze during collection. In fact, some of the material may fall into the surgical site, thereby risking patient infection. Still further, several pieces of gauze may be required for the procedure, which is not only wasteful, but also requires that each piece of gauze must be accounted for.

Although this widely used procedure is simple and doesn't require much additional equipment, the collected bone and tissue is exposed to contaminants, such as those in the air, on the gauze, and/or on the hands of the surgeon or surgical assistant. This may be an acceptable risk if the collected material is to be discarded, but is unacceptable if the material may be reused. For example, tissue may be removed from the collected bone fragments, and the fragments may then be processed for use as a bone graft in a spinal fusion procedure. Implanting contaminated bone into a patient, whether the bone is used as an allograft or autograft, increases the chances of rejection, infection, and other complications.

It is therefore desired to provide a device and method for removing biological material, such as bone and/or tissue, from a surgical tool (referred to herein as “cleaning” a surgical tool) and also for storing collected biological material, such as bone and/or tissue, in a sterile environment, thereby reducing the likelihood of contamination of the collected material. It is further desired to provide a system and method for easily collecting bone and tissue without looking away from a surgical procedure at hand.

### SUMMARY OF THE INVENTION

The present invention advantageously provides a method and system for cleaning a surgical tool and storing bone and tissue. In one embodiment, a system for collecting biological material may include a housing, the housing including an entry opening and a brush assembly chamber, the brush assembly chamber in communication with the entry opening. The system may also include a brush assembly disposed within the brush assembly chamber and a storage container in communication with the brush assembly chamber. The housing may further include a ramp face, which may define the entry opening. Further, the ramp face may be substantially trapezoidal and may include a lip that has a first portion, a second portion, a third portion, and a fourth portion. The first and fourth portions may be substantially parallel. The ramp face may further define a central ridge, a substantially planar portion, and a concave portion. The system may further include a motor and a power source, and the housing may further include a motor housing component and a base component. The system may include a first storage container and a second storage container, each of which being in communication with the brush assembly chamber. Alternatively, the system may include a first brush assembly chamber, a second brush assembly chamber, a first brush assembly and a second brush assembly, a first entry opening that is defined by the ramp face, and a second entry opening that is defined by the ramp face. The second brush assembly may be located within the second brush assembly chamber, the second entry opening may be in communication with the second brush assembly chamber, and the second brush assembly chamber may be in communication with the second storage container. The ramp face may define a central ridge disposed between the first entry opening and the second entry opening, the central ridge extending between the first portion of the lip and the fourth portion of the lip. Further, the central ridge may define an indentation along at least a portion of the longitudinal axis of the central ridge. The ramp face may define a first entry portion and a second entry portion that are separated at least in part by the central ridge, and each of the first and second entry portions may include a first area that is substantially planar and a second portion that is concave. The housing may further include at least one connection outlet, each storage container being removably attachable to a connection outlet. Each container may include a first open end, a second closed end, and an access opening. The first end may be removably attachable to the connection outlet, which may be in communication with the brush assembly chamber.

In another embodiment, a system for collecting bone and tissue may generally include a housing, the housing defining a first entry opening and a second entry opening, a first brush assembly chamber and a second brush assembly chamber, the first brush assembly chamber being in communication with the first entry opening and the second brush assembly chamber being in communication with the second entry opening, and a ramp face, the ramp face defining the first and second entry openings and a central ridge disposed between the first and second entry openings. The system may further include a first brush assembly disposed within the first brush assembly chamber and a second brush assembly disposed within the second brush assembly chamber, a first storage container in communication with the first brush assembly chamber and a second storage container in communication with the second brush assembly chamber, and a motor in communication with the first and second brush assemblies, activation of the motor rotating each of the first and second brush assemblies.

A method for collecting biological material from a surgical device may generally include inserting a distal end of a surgical device into an entry opening of a collection device, the distal end of the surgical device including biological material, rotating a brush assembly within a brush assembly chamber, the brush assembly and brush assembly chamber being disposed within the collection device, the brush assembly chamber being in communication with the entry opening, and removing biological material from the distal end of the surgical device with the rotating brush assembly. The method may further include collecting the removed biological material in a container that is removably attached to the collection device.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows a front view of a first embodiment of a surgical tool cleaner and bone and tissue collection device;

FIG. 2 shows a side perspective view of the first embodiment of a surgical tool cleaner and bone and tissue collection device;

FIG. 3 shows a rear view of the first embodiment of a surgical tool cleaner and bone and tissue collection device;

FIG. 4 shows a top view of the first embodiment of a surgical tool cleaner and bone and tissue collection device;

FIG. 5A shows a first embodiment of a brush assembly;

FIG. 5B shows a second embodiment of a brush assembly;

FIG. 6 shows a front perspective view of a second embodiment of a surgical tool cleaner and bone and tissue collection device;

FIGS. 7A-7D show steps in a method of harvesting bone and tissue and storing the collected material in a surgical tool cleaner and bone and tissue collection device;

FIG. 8 shows a first embodiment of a handheld surgical tool cleaner and bone and tissue collection device; and

FIG. 9 shows a second embodiment of a handheld surgical tool cleaner and bone and tissue collection device.

### DETAILED DESCRIPTION OF THE INVENTION

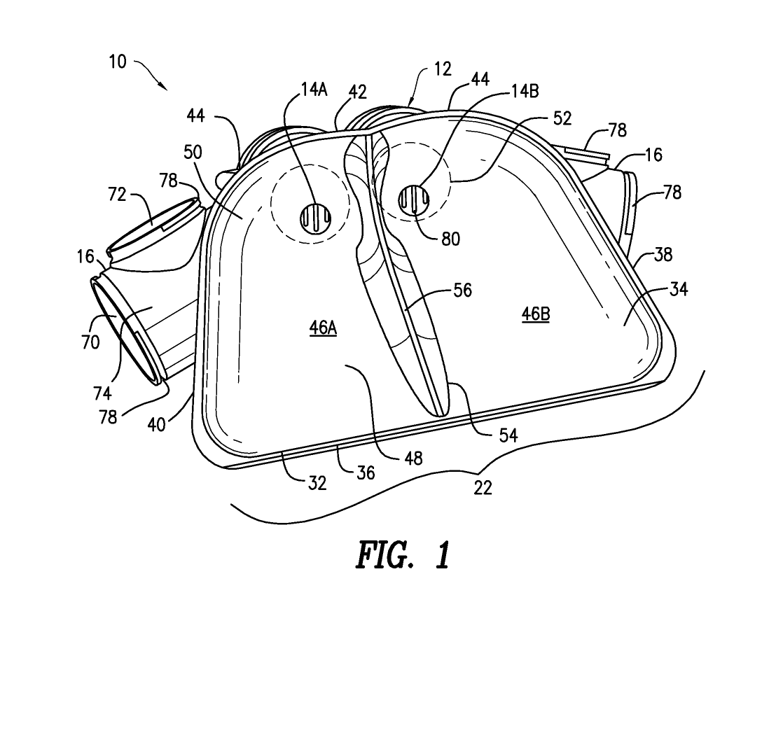

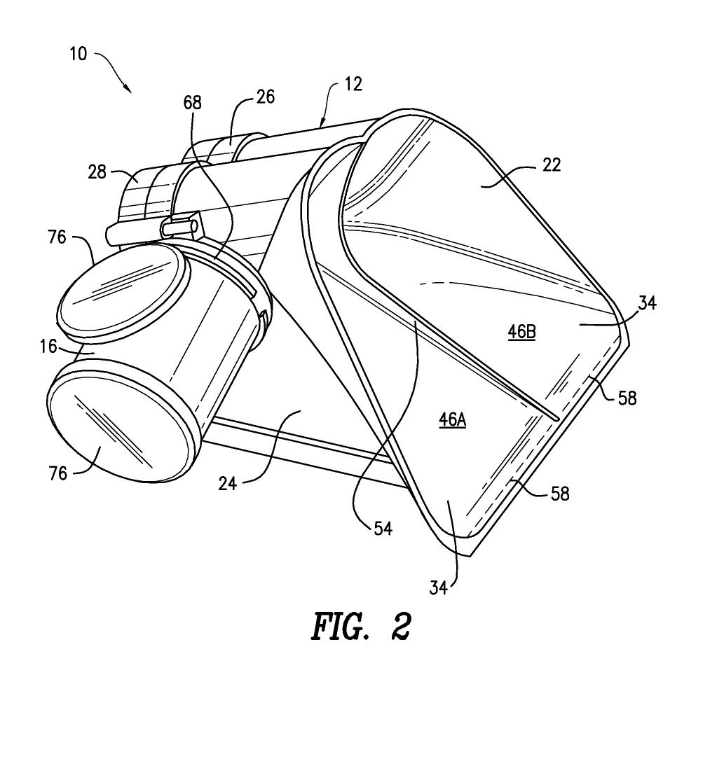

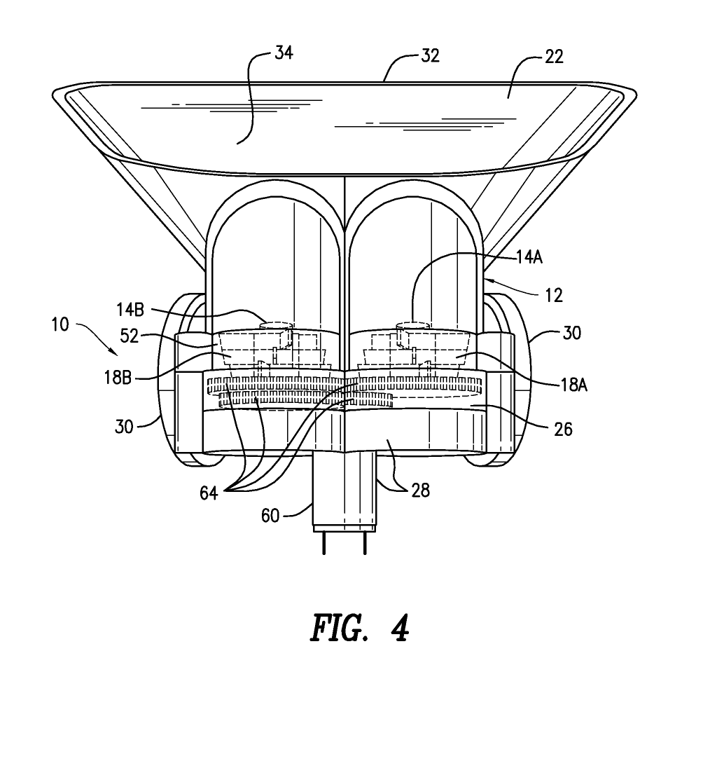

The present invention advantageously provides a method and system for cleaning a surgical tool and storing bone and tissue. Referring now to the drawing figures in which like reference designations refer to like elements, a first embodiment of a surgical tool cleaner and bone and tissue collection device is shown in FIGS. 1-4 and generally designated as “10.” Of note the device components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein. Moreover, while certain embodiments or figures described herein may illustrate features not expressly indicated on the other figures or embodiments, it is understood that the features and components of the system and devices disclosed herein are not necessarily exclusive of each other and may be included in a variety of different combinations or configurations without departing from the scope and spirit of the invention.

Continuing to refer to FIGS. 1-4, the device 10 may generally include a housing 12, a plurality of entry openings 14, a plurality of material storage containers 16, a plurality of brush assemblies 18, and a power source 20. The housing 12 may include an entry ramp 22, a base component 24, a gear housing component 26, a motor housing component 28, and a plurality of material storage container connection outlets 30. The housing 12 may be composed of a rigid plastic, such as, for example, polyethylene. Further, the device 10 may be composed of cost- effective materials such that the device is suitable for single-use applications. Alternatively, the device 10 may be composed of plastics, metals, polymers, and/or other materials suitable for multiple-use applications.

The entry ramp 22 may include a lip 32 that surrounds the ramp face 34. The lip 32 may define a substantially horizontal first portion 36, a substantially vertical second portion 38, a substantially vertical third portion 40, and a substantially horizontal fourth portion 42. The second 38 and third 40 portions may each meet the first portion 36 at an angle that is approximately 90° or less, and the second 38 and third 40 portions may each meet the fourth portion 42 at an angle that is approximately 90° or more. So, the lip 32 may define a rectangular or trapezoidal perimeter around the ramp face 34. Optionally, the fourth portion 42 may include two curved areas 44 that meet in approximately the middle of the fourth portion 42 of the lip 32.

The ramp face 34 may define a plurality of entry portions 46, such as a first entry portion 46A and a second entry portion 46B as shown in FIGS. 1-4. Each of the first and second entry portions 46A, 46B may include a first area 48 that is substantially planar and a second area 50 that is concave. In general, each of the first and second entry portions 46A, 46B may be substantially frustoconical, the first and second entry portions 46A, 46B defining a first entry opening 14A and second entry opening 14B, respectively. Each entry opening 14 may be in communication with a brush assembly chamber 52. Additionally, the ramp face 34 may define a central ridge 54 that has a longitudinal axis that substantially extends from the first lip portion 36 to the fourth lip portion 42, between the first and second entry portions 46A, 46B. At least a portion of the ridge 54 may define an indentation 56 that extends along the longitudinal axis of the ridge 54. The central ridge 54 may physically separate the first and second entry portions 46A, 46B of the ramp face 34, so that a surgical tool is less likely to be mistakenly inserted into the wrong entry opening.

The ramp face 34 and/or lip 32 may include a pressure and/or movement sensor that allows for the automatic activation of the motor 60 when a user touches the ramp face 34 and/or lip 32 with the surgical tool. Thus, the motor 60 may be inactive during the times when the user is performing the surgical procedure, and may be activated only when the device 10 is actively used to collect bone and tissue from a surgical tool (that is, when a tool is in contact with or proximate the ramp face 34 and/or entry opening 14).

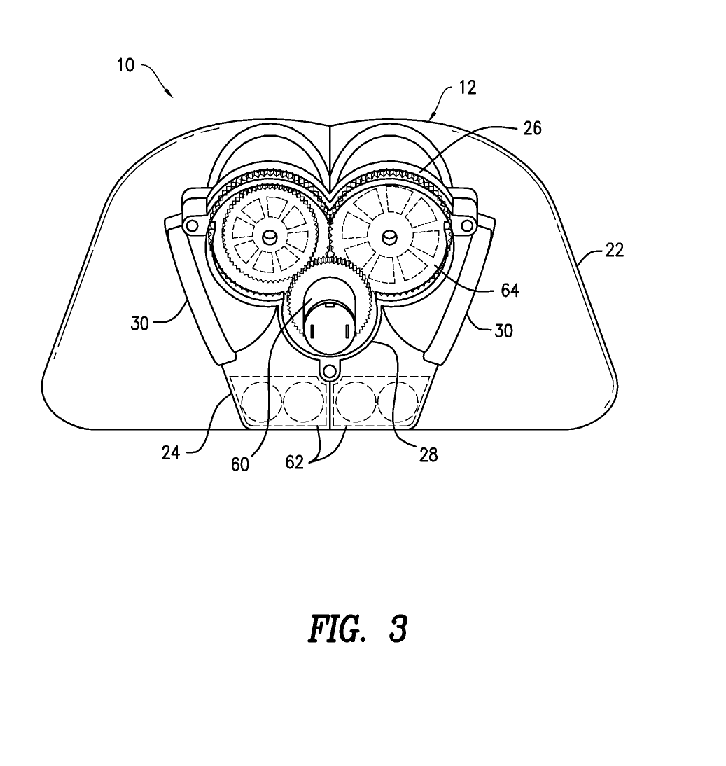

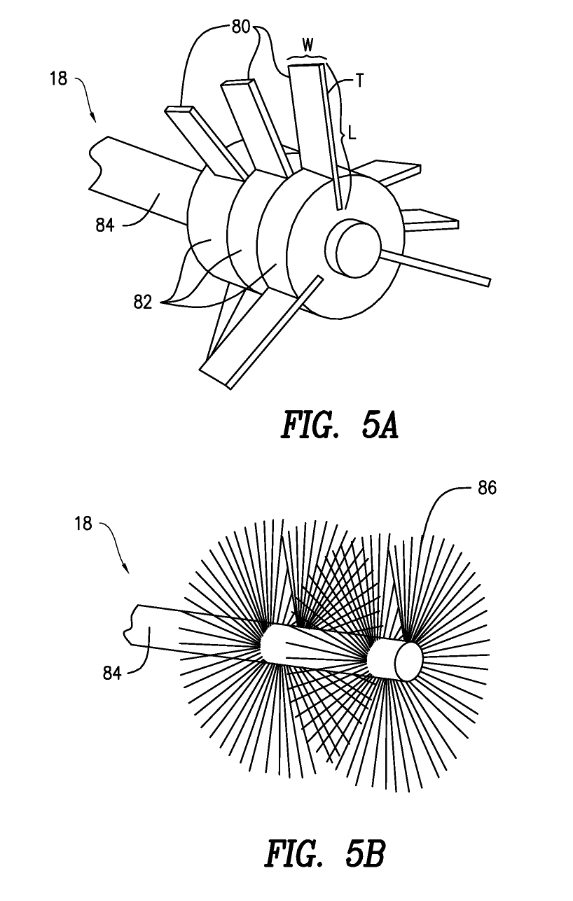

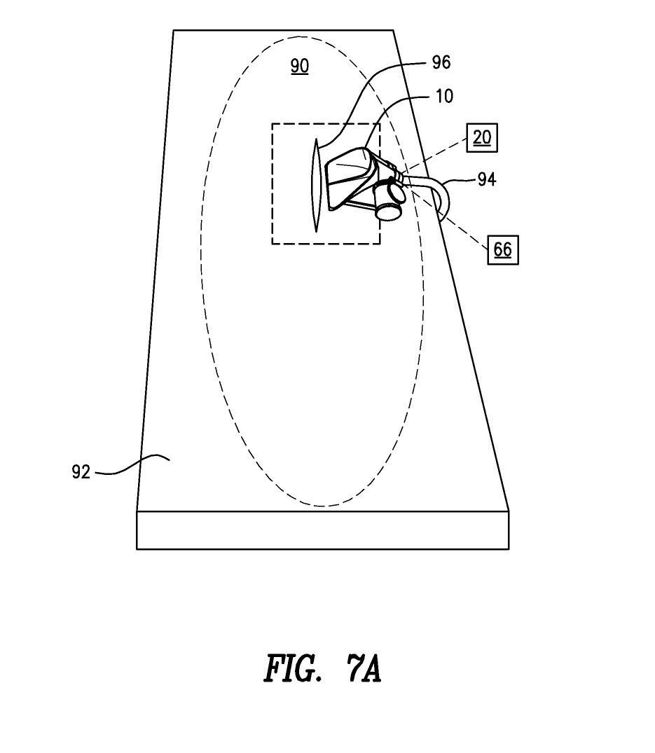

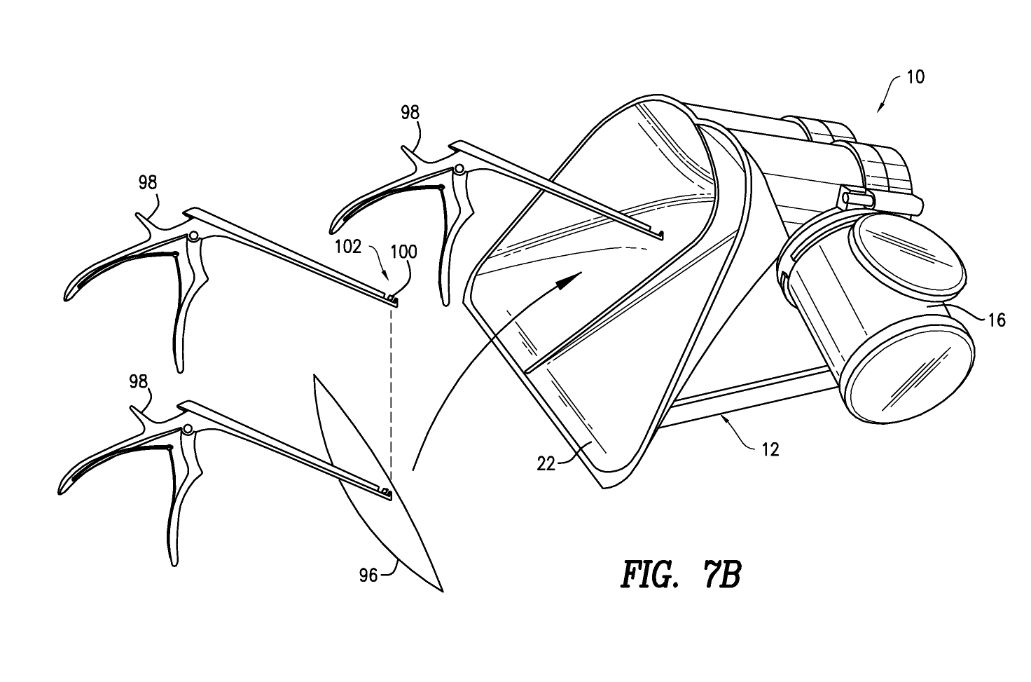

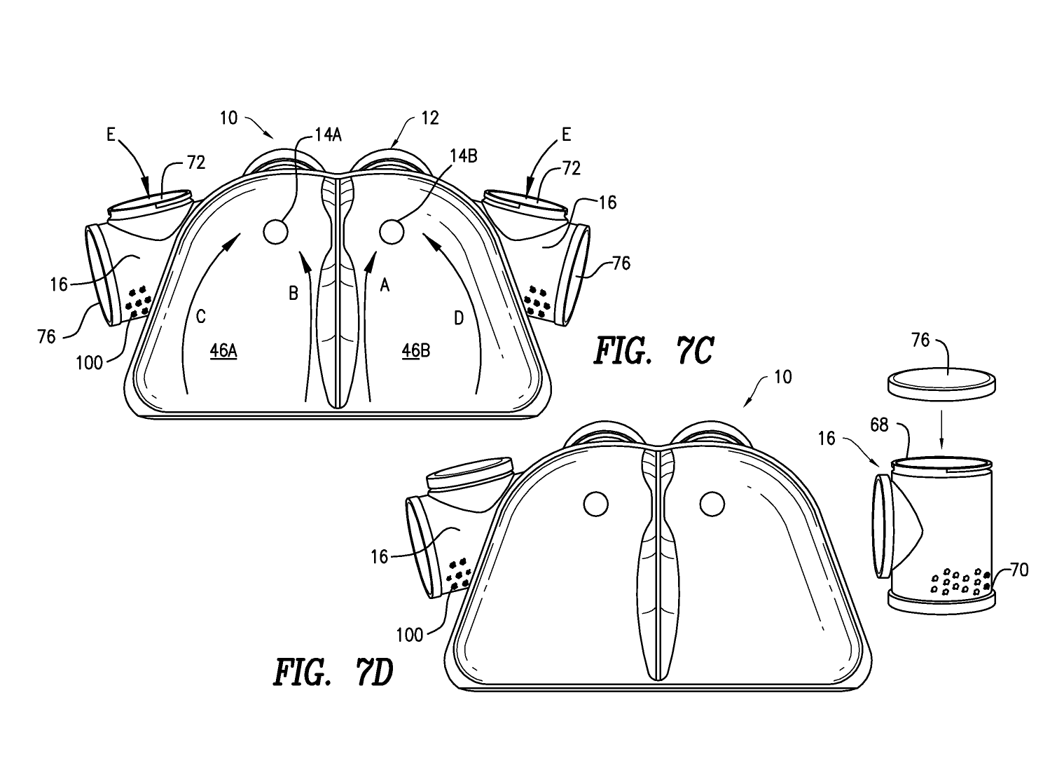

The base component 24 of the housing 12 may define at least one battery storage compartment 62, and one or more batteries contained within may serve as a primary or backup power source 20. The batteries may provide power to a motor 60 located within the motor housing component 28, which may, in turn, drive one or more gears 64 located within the gear housing component 26 (for example, as shown in FIG. 3). As a non-limiting embodiment, the motor 60 may be one such as Athlonix 16N78 (Portescap, West Chester, Pa.). Optionally, the motor 60 may be in communication with a switch that may reverse the rotation of a motor shaft and, thus, the brush assemblies 18. For example, the switch may be in communication with a foot pedal or touchpad 66 that is easily operable by the user without taking the user's attention away from the surgical procedure at hand (for example, as shown in FIG. 7A). The one or more gears 64 may be in mechanical communication with the plurality of brush assemblies 18 (as shown in FIGS. 5A and 5B). Although four gears 64 are shown, the device 10 may include any number of gears sufficient to translate rotation of a shaft connected to the motor 60 into rotation of the brush assemblies 18. Additionally or alternatively, the motor 60 may be in communication with a power source 20 such as a generator or wall outlet. Activation of the motor 60 may drive the one or more gears 64, thereby causing the plurality of brush assemblies 18 to rotate. Each of the plurality of brush assemblies 18 may rotate about an axis that is substantially parallel to a longitudinal axis of the brush assembly chamber 52 in which the brush assembly 18 is located. Further, each of the plurality of brush assemblies 18 may rotate in the same or different directions. As a non-limiting example, a first brush assembly 18A may rotate in a clockwise direction, whereas a second brush assembly 18B may rotate in a counterclockwise direction. Exemplary brush assemblies 18 are shown and described in FIGS. 5A and 5B.

Continuing to refer to FIGS. 1-4, each of the plurality of material storage containers 16 may be removably attachable to device 10 at a connection outlet 30. As a non-limiting example, the device 10 may include a first storage container 16A and a second storage container 16B. Each brush assembly chamber 52 may include a first opening defined by the ramp face 34, such as the entry opening 14, and a second opening at least partially defined by a connection outlet 30. The inner diameter of the brush assembly chamber 52 may be greater than the diameter of the entry opening 14, and the entry opening 14 may be located such that only a small portion of the brush assembly is accessible by a surgical tool or device when inserted into the entry opening 14. Thus, bone and tissue swept or dislodged by each brush assembly 18 from a surgical tool may be directed from the brush assembly chamber 52 into the connection outlet 30 and into the material storage container 16. Each of the plurality of material storage containers 16 may be substantially cylindrical with a substantially circular cross section, although it will be understood that the containers 16 may have any other shape suitable for use with the housing 12 and that can contain bone fragments and other material. Each container 16 may include an open first end 68, a closed second end 70, and an access opening 72 located in a portion of the container wall 74. Although the closed second 70 end may be openable (and is shown as being open in, for example, FIG. 1), it is referred to as being closed because in use the second end 70 may be closed by a removably attachable lid 76. Alternatively, the second end 70 may be closed during manufacturing. For example, the second end 70 may be an integrated portion of the container wall 74. Further, each of the plurality of material storage containers 16 may be in communication to one of the material storage container connection outlets 30. At least the first end 68 of each container 16 may define a threaded portion 78 that is matably connectable with a connection outlet 30. The second end 70 and the access opening 72 may also each define a threaded portion 78. The amount of threading on the container 16 and the connection outlet 30 may be such that the container 16 becomes connected to the housing 12 with an approximately 90° to approximately 180° rotation in either a clockwise or counterclockwise direction, depending on the configuration of the threading. When a container 16 is connected to the housing 12, the access opening may be located on an upper portion of the container 16 relative to the housing 12. The access opening may be sealed with a lid 76, which may be removed if the user wants to deposit a larger piece of bone and/or tissue into the container 16 while bypassing the brush assemblies 18.

The material storage containers 16 may be stored in a sterile environment prior to being attached to the device 10, such that sterility of the containers is maintained. Further, bone and tissue fragments that are collected from a surgical tool by the device 10 may be protected from contamination while in the containers. Optionally, each container 16 may include a UV light source for killing or inhibiting growth of microbes during storage of the bone and tissue fragments. Additionally or alternatively, an antiseptic powder, solution, or liquid may be added to the bone and tissue within the containers.

In the embodiment shown in FIGS. 1-4 having a first entry opening 14A and a second entry opening 14B, bone and tissue fragments may be selectively collected within either of a first 16A or second 16B material storage container. For example, at least a portion of the surgical tool may be inserted through the first entry opening 14A and into the first brush assembly chamber 52A, within which the first brush assembly 18A may sweep bone and tissue from the surgical tool into the first material storage container 16A. Likewise, the surgical tool may be inserted through the second entry opening 14B and into the second brush assembly chamber 52B, within which the second brush assembly 18B may sweep bone and tissue from the surgical tool into the second material storage container 16B. In this way, a user may collect viable bone and tissue in one container and may collect nonviable, unusable, and/or waste bone and tissue into a second container. Further, the shape of the ramp face 34 and central ridge 54 may physically direct the surgical tool into one of the entry openings 14A, 14B, thereby allowing the user to insert the tool into the appropriate entry opening with tactile feedback and without visual feedback. That is, the user may collect the bone and tissue without taking his or her eyes away from the surgical procedure at hand. This may be referred to as a “blind technique.” Additionally, the device 10 may allow the surgeon to quickly and easily collect material without having to involve an assistant, and may also increase the productivity of the surgical assistant, for example, by eliminating the need for bone collection in a piece of gauze or lap sponge. Still further, this technique allows for a more precise method of bone and tissue collection that is less subject to human error, as this consistent method of collection is controlled by the user.

Referring now to FIGS. 5A and 5B, a first embodiment and second embodiment of a brush assembly 18 are shown. In the first embodiment, the brush assembly 18 may include a plurality of resiliently deformable paddles or blades 80 coupled to a plurality of annular elements 82. As a non-limiting example, the brush assembly 18 may include a shaft 84 and a plurality of annular elements 82, and each annular element may include a plurality of paddles. As a non-limiting example, each annular element 82 may include three paddles 80, the paddles 80 radially symmetrically spaced at 0°, 120°, and 240° about the shaft. Further, the paddles 80 of each annular element 82 may be offset such that no paddles 80 of adjacent annular elements 82 lie at the same radial direction (as shown in FIG. 5A). For example, the paddles 80 of adjacent annular elements 82 may be offset by between approximately ±5° and approximately ±60°. Each paddle 80 may be composed of a material, such as latex, rubber, or other flexible materials, that has a durometer that is less than that of the surgical tool and the plurality of annular elements 82, thus allowing the paddles 80 to sweep bone and tissue fragments from a surgical tool without damaging the tool or exerting excessive pressure on the annular elements during bone and tissue collection. Further, each paddle 80 may have a length L, width W, and thickness T, with the length L being greater than the width W and the width W being greater than the thickness T.

As shown in FIG. 5B, a second brush assembly 18 embodiment may include a plurality of bristles 86 instead of paddles 80. The bristles 86 may be spirally arranged about an annular element 82 that is coupled to a shaft 84. Like the paddles, however, the bristles 86 may be composed of a resiliently deformable material having a durometer less than the surgical tool and annular element 82.

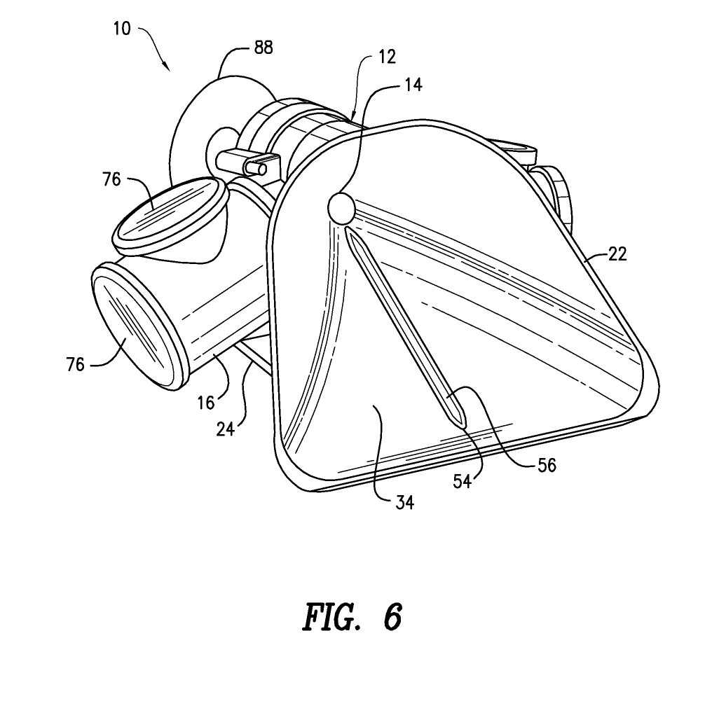

Referring now to FIG. 6, a second embodiment of a surgical tool cleaner and bone and tissue collection device 10 is shown. The device 10 of FIG. 6 may be substantially similar to the device 10 of FIGS. 1-4, in form and function. However, unlike the first embodiment of a surgical tool cleaner and bone and tissue collection device 10 shown in FIGS. 1-4, the device 10 of FIG. 6 may include only one entry opening 14 defined by the ramp face 34. Similarly, the device 10 of FIG. 6 may include only one brush assembly 18, brush assembly chamber 52, and material storage container 16. The brush assembly 18 may be as is shown and described in FIG. 5A or 6B. Additionally, the device 10 may not include a plurality of gears or a gear housing component 26. Rather, the motor shaft may be in direct communication with the brush assembly 18. In fact, the motor shaft may be the brush assembly shaft 84.

Continuing to refer to FIG. 6, the ramp face 34 may define a first area 48 that is substantially planar and a second area 50 that is concave. As a whole, the ramp face 34 may be roughly frustoconical, the shape of the ramp and central ridge 54 directing a surgical tool toward the entry opening 14. This configuration may be used for procedures that involve the removal of bone and tissue from the patient without the intention of saving the material for later use, such as a revision procedure. Although the device 10 is shown in FIG. 6 as including only one material storage container 16, the device 10 may also be configured to accept more than one container 16, such as shown and described in FIGS. 1-4. In this case, the motor 60 may be in communication with a switch that may allow the user to select the rotation of the brush assembly 18, and thus the container 16 into which the bone and tissue may be swept. For example, usable bone and tissue fragments may be collected into one container, whereas refuse bone and tissue may be collected into another container by changing the direction of the brush assembly 18 rotation as appropriate. The switch may be in communication with a foot pedal or touchpad 66 that is easily operable by the user without taking the user's attention away from the surgical procedure at hand.

As is also shown in FIG. 6, the device 10 may optionally include a handle 88. Although not shown in FIGS. 1-4, the device 10 having two entry openings may also include a handle. The handle 88 may be coupled to the housing 12, and may provide the user with greater control over the location and use of the device 10. It will be understood that the handle 88 may have any suitable shape, size, configuration, or means of attachment to the housing.

Referring now to FIGS. 7A-7D, steps in a method of harvesting bone and tissue and storing the collected material in a surgical tool cleaner and bone and tissue collection device 10 are shown. As shown in FIG. 7A, a patient 90 (depicted as an oval for simplicity) may be positioned on a table or platform 92, and the surgical tool cleaner and bone and tissue collection device 10 may be coupled to a flexible arm 94 proximate the surgical site 96. For example, the flexible arm 94 may be coupled to the table 92 or to a stand located near the table, and the flexible arm 94 may be positioned such that the ramp face 34 of the device 10 is located proximate the surgical site 96, such as a portion of the patient's spinal column. Affixing the device 10 to a flexible arm, the table, and/or a base may provide stability to the device 10 and ensure that the device 10 remains at a location convenient for the user.

As shown in FIG. 7B, a surgical tool 98, such as a Kerrison Rongeur tool, may be used to collect bone and/or tissue fragments 100 from the patient 90 and transfer the collected material to the device 10. As shown in FIG. 7C, the distal end 102 of the tool 98 may be advanced along the central ridge 54 and/or the ramp face 34 toward an entry opening 14. The shape of the ramp face 34 and central ridge 54 may help direct the tool 98 toward an entry opening 14, without requiring the user to direct his or her attention away from the surgical procedure at hand. For example, a device 10 may travel up the central ridge 54 to either the first 14A or second entry opening 14B (as shown in FIG. 7C as paths A and B), along the ramp face 34 to a concave portion 50 of the first or second entry portions 46A, 46B (as shown in FIG. 7C as paths C and D), or along any other path that leads to the target entry opening 14. Additionally, the user may insert bone fragments, such as larger bone fragments that would not fit through the entry openings, directly into a material storage container 16 through that container's access opening 72. Although a device 10 having two entry openings 14A, 14B is shown in FIGS. 7A-7D, it will be understood that a device 10 having a single entry opening 14 may also be used (as shown and described in FIG. 6). Within the brush assembly chamber 52, the brush assembly 18 may automatically clean (that is, remove material from) the distal end 102 of the tool 98.

As shown in FIG. 7D, once a desired amount of bone and/or tissue 100 has been collected from the patient 90, one or more material storage containers 16 may be removed from the device 10 and the open first end 68 may be sealed with a removable lid 76. For example, the lid 76 may define a threading that is matably connectable to the threading of the open end 68 of the container 16. The sealed container 16 may then be stored or attached to a bone mill for bone cleaning and fragmentation, which may be required in order to prepare the collected bone, for example, for use as a bone graft in a spinal fusion procedure.

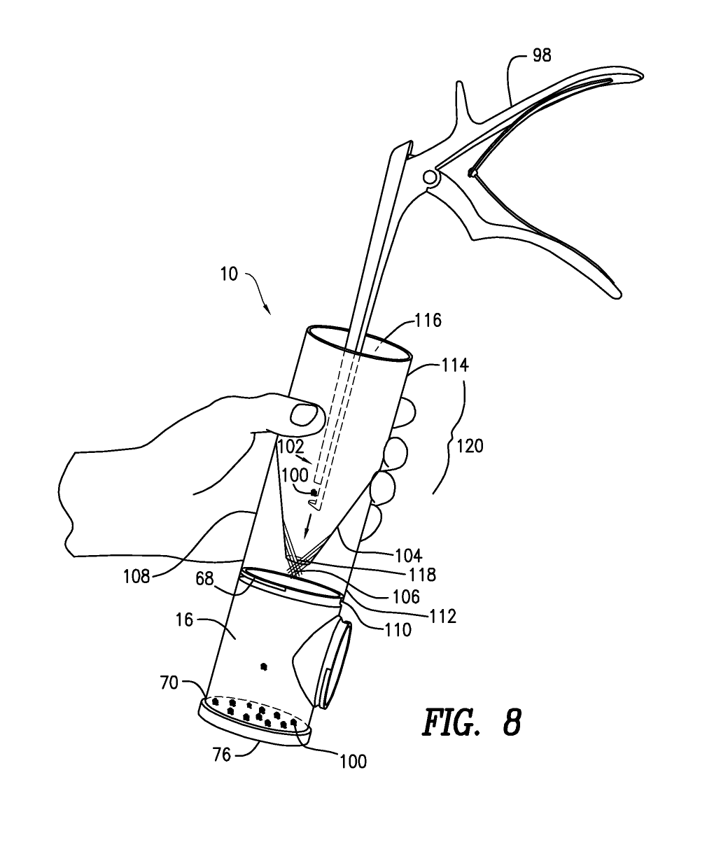

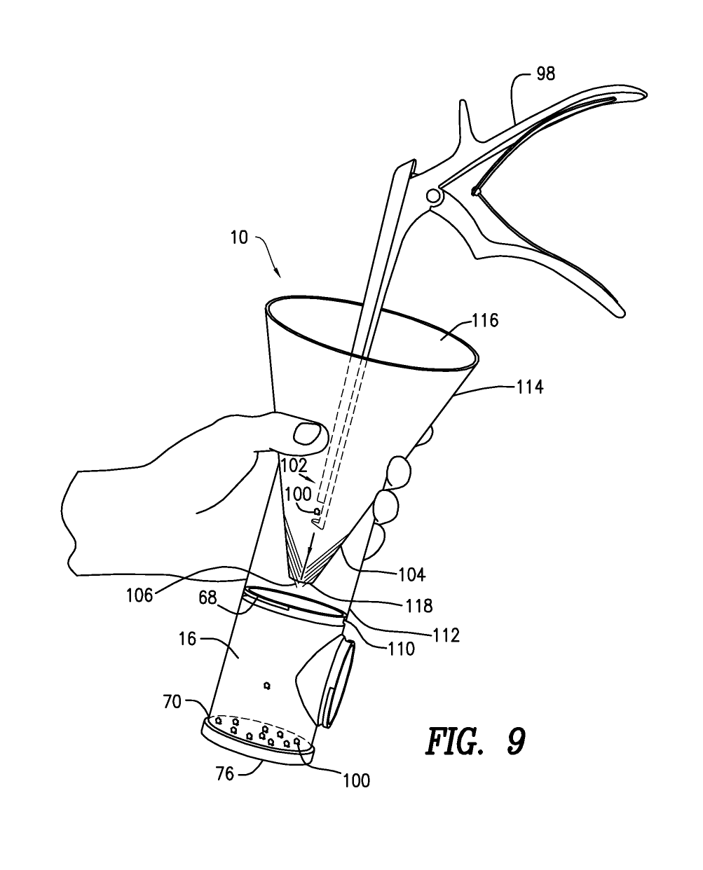

Referring now to FIGS. 8 and 9, a first embodiment and a second embodiment of a handheld surgical tool cleaner and bone and tissue collection device 10 are shown. The devices 10 shown in FIGS. 8 and 9 are generally similar to each other, with each device 10 including a conical portion 104, a brush assembly 106, a body portion 108, and a material storage container 16. The body portion 108 may define a collection outlet 110 at a proximal end 112 of the device 10. The conical portion 104 may define a first opening 116 at a distal end of the device 10 and a second opening 118 within the body portion 108 of the device 10, the first opening 116 having a larger diameter than the second opening 118. Further, the second opening 118 may include a brush assembly 106. The brush assembly 18 may include a plurality of bristles, and the bristles may be directed toward the material storage container 16, which may be coupled to the proximal end 112 of the device 10. The bristles may be overlapping (for example, as shown in FIG. 8) or may be non-overlapping (for example, as shown in FIG. 9). The bristles may be immovably affixed to the second opening 118 of the conical portion 104 as shown in FIGS. 8 and 9, or they may be part of a rotatable brush assembly, which may be similar to those shown and described in FIGS. 5A and 5B. In this case, the brush assembly 18 may be in mechanical communication with a motor 60 that rotates the brush assembly in a clockwise or counterclockwise direction. As a non-limiting example, the brush assembly 18 may be rotatable about the longitudinal axis of the device 10 or about an axis that is substantially orthogonal to the longitudinal axis of the device 10. Although not shown, a plurality of bristles may also be located within the conical portion 104 itself, and not just as part of the brush assembly 106. Additionally, it will be understood that any configuration of bristles may be used that is suitable to dislodge biological materials such as bone and/or tissue from a surgical tool.

The material collection container 16 may be as shown and described in FIGS. 1-7D, and may be removed from the device 10 for storage or further use once the bone and tissue has been collected. The collection outlet 110 the body portion 108 of the device 10 may be matably connectable to the open end 68 of the container 16, as shown and described in FIGS. 1-7D. Bone and tissue may be collected from the patient and immediately passed directly into the device 10 and sterile storage containers, thereby reducing or eliminating the risk that collected material will become contaminated. As a result, if the material is later reused in a procedure, for example, when used as a bone graft in a spinal fusion procedure, there may be a better chance that the bone will remain viable and a decreased risk that infection may develop at the fusion site.

The conical portion 104 of the embodiment shown in FIG. 8 may include a cylindrical portion 120 that has the same inner and outer diameter as the body portion 108 of the device 10. That is, the device 10 as a whole may have a cylindrical shape, with the conical portion 104 being disposed entirely within the body portion 108. Alternatively, as shown in FIG. 9, the conical portion 104 of the device 10 may include a first opening 116 having a diameter that is greater than the diameter of the body portion 108 of the device 10.

During use, a surgical tool 98 may be inserted into the first opening 116 of the conical portion 104 and through the second opening 118 of the conical portion 104, such that the plurality of bristles of the brush assembly 106 (or 18) sweeps bone and tissue 100 from the surgical tool 98 and into the material collection container 16. Like the ramp face 34 of the devices shown and described in FIGS. 1-7D, the conical portion 104 of the device 10 of FIGS. 8 and 9 may physically direct the surgical tool 98 toward the brush assembly 106, such that a user may collect bone and tissue within the device 10 without taking his or her attention away from the surgical procedure at hand. The container 16 may then be removed from the device 10 and stored or used in procedures for further processing collected bone and/or tissue.

Although not shown, a material storage container may be affixed or coupled to a glove that is wearable by the user. The glove may include one or more tool cleaning portions, such as an area having a plurality of bristles on each finger portion of the glove. The glove may also include a low-friction pathway from the tool cleaning portions into the container. In use, the one or more tool cleaning portions may dislodge bone and/or tissue material form a surgical tool, such as a Kerrison Rongeur tool, which material may then fall along the low-friction pathway into the container.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A system for collecting biological material, the system comprising:
a housing, the housing including an entry opening and a brush assembly chamber, the brush assembly chamber in communication with the entry opening;
a brush assembly disposed within the brush assembly chamber; and
a storage container in communication with the brush assembly chamber.

2. The system of claim 1, wherein the housing further includes an entry ramp defining a ramp face, the ramp face defining the entry opening.

3. The system of claim 2, wherein the ramp face is substantially trapezoidal.

4. The system of claim 3, wherein the ramp face includes a lip that has a first portion, a second portion, a third portion, and a fourth portion.

5. The system of claim 4, wherein the first and fourth portions are substantially parallel.

6. The system of claim 1, the system further comprising a motor and a power source.

7. The system of claim 6, wherein the housing further includes a motor housing component and a base component.

8. The system of claim 7, wherein the system further comprises a motor located within the motor housing component and a power source located within the base component.

9. The system of claim 2, wherein the ramp face further defines a central ridge, a substantially planar portion, and a concave portion.

10. The system of claim 2, wherein the storage container is a first storage container, the system further comprising a second storage container.

11. The system of claim 10, wherein the second storage container is in communication with the brush assembly chamber.

12. The system of claim 10, wherein the entry opening is a first entry opening, the brush assembly chamber is a first brush assembly chamber, and the brush assembly is a first brush assembly, the system further comprising a second entry opening, a second brush assembly chamber, and a second brush assembly.

13. The system of claim 12, wherein the second brush assembly is located within the second brush assembly chamber, the second entry opening is in communication with the second brush assembly chamber, and the second brush assembly chamber is in communication with the second storage container.

14. The system of claim 13, wherein the ramp face further defines the second entry opening.

15. The system of claim 14, wherein the ramp face further defines a central ridge disposed between the first entry opening and the second entry opening, the central ridge extending between the first portion of the lip and the fourth portion of the lip.

16. The system of claim 15, wherein the central ridge defines an indentation along at least a portion a longitudinal axis of the central ridge.

17. The system of claim 15, wherein the ramp face further defines a first entry portion and a second entry portion that are separated at least in part by the central ridge, each of the first and second entry portions including a first portion that is substantially planar and a second portion that is concave.

18. The system of claim 1, wherein the housing further includes a connection outlet, the storage container being removably attachable to the connection outlet.

19. The system of claim 18, wherein the container includes a first open end, a second closed end, and an access opening, the first end being removably attachable to the connection outlet, the connection outlet in communication with the brush assembly chamber.

20. The system of claim 1, further comprising a flexible arm, the housing being attached to the flexible arm.

21. The system of claim 1, wherein the biological material is bone.

22. A system for collecting bone and tissue, the system comprising:
a housing, the housing defining:
a first entry opening and a second entry opening;
a first brush assembly chamber and a second brush assembly chamber, the first brush assembly chamber being in communication with the first entry opening and the second brush assembly chamber being in communication with the second entry opening; and
an entry ramp, the entry ramp defining the first and second entry openings and a central ridge disposed between the first and second entry openings;
a first brush assembly disposed within the first brush assembly chamber and a second brush assembly disposed within the second brush assembly chamber;
a first storage container in communication with the first brush assembly chamber and a second storage container in communication with the second brush assembly chamber; and
a motor in communication with the first and second brush assemblies, activation of the motor rotating each of the first and second brush assemblies.

23. A method for collecting biological material from a surgical device, the method comprising:
inserting a distal end of a surgical device into an entry opening of a collection device, the distal end of the surgical device including biological material;
rotating a brush assembly within a brush assembly chamber, the brush assembly and brush assembly chamber being disposed within the collection device, the brush assembly chamber being in communication with the entry opening; and
removing biological material from the distal end of the surgical device with the rotating brush assembly.

24. The method of claim 22, the method further comprising:
collecting the removed biological material in a container that is removably attached to the collection device.
